(19) United States
(12) Patent Application Publication
(10) Pub. No.: US 2021/0267694 A1
(43) Pub. Date: Sep. 2, 2021

(54) INCORPORATING NEW LOCATION READINGS TO OLD MODELS

(71) Applicant: NAVIX INTERNATIONAL LIMITED, Road Town, Tortola (VG)

(72) Inventor: Eli DICHTERMAN, Haifa (IL)

(73) Assignee: Navix International Limited, Road Town, Tortola (VG)

(21) Appl. No.: 17/257,600

(22) PCT Filed: Jul. 4, 2019
(86) PCT No.: PCT/IB2019/055733

§ 371 Date: Jan. 4, 2021

Related U.S. Application Data
(60) Provisional application No. 62/807,851, filed on Feb. 20, 2019.
(60) Provisional application No. 62/693,942, filed on Jul. 4, 2018.

(51) Int. Cl. A61B 34/20; A61B 5/06

(52) U.S. Cl. CPC A61B 34/20; A61B 5/068

(57) ABSTRACT

A method of estimating a location of a catheter in a heart chamber with respect to a model is provided. The model is generated based on readings received from the catheter. The method, in some embodiments, includes receiving readings from the catheter when the catheter is in the location to be estimated; and estimating the location based on the location readings received together with a completeness indicator, indicative of a completeness of the model. Examples of completeness indicators include elapsed time and variance in the model data.

### FIELD AND BACKGROUND OF THE INVENTION

The present invention, in some embodiments thereof, relates to the field of navigation of body cavities by intra-body probes, and more particularly, to reconstruction of body cavity shape from measurements by intra-body probes.

The present disclosure, in some embodiments thereof, relates to estimating one or more intra-body locations based on electrical location readings taken at or near the location being estimated; for example, for navigation of a tool carrying a sensor that takes the location reading, and/or imaging a volume from which the location readings are taken.

Several medical procedures in cardiology and other medical fields comprise the use of intra-body probes such as catheter probes to reach tissue targeted for diagnosis and/or treatment while minimizing procedure invasiveness. Early imaging-based techniques (such as fluoroscopy) for navigation of the catheter and monitoring of treatments continue to be refined, and are now joined by techniques such as electromagnetic field-guided position sensing systems.

Earlier patent applications by the Applicant, relating to electromagnetic field-guided position sensing and/or imaging were published as international patent application publications Nos. WO2018/078540 and WO2018/130974.

### SUMMARY OF THE INVENTION

Some examples of embodiments of the invention follows. It should be noted that some embodiments of the invention include features from multiple examples and/or can include fewer than all features described in one example.

Example 1. A method of estimating a location of a catheter in a body part with respect to a model of the body part, wherein the model was generated based on model location readings received from the catheter before the catheter was in the location to be estimated, the method comprising:
receiving current location readings from the catheter when the catheter is in the location to be estimated; and
estimating the location based on the current location readings received, and a completeness indicator, indicative of a completeness of the model.

Example 2. The method of example 1, wherein the body part is a heart chamber.

Example 3. The method of example 1 or example 2, wherein the completeness indicator is based on the time that elapsed from when the model generation started and the time the location readings were received from the location to be determined.

Example 4. The method of example 1 or example 2, wherein the completeness indicator is based on variance in model location readings.

Example 5. The method of example 4, wherein the completeness indicator indicates greater completeness as overall variance increases.

Example 6. The method of any one of examples 4-5, wherein the completeness indicator indicates greater completeness as a rate of change in overall variance decreases.

Example 7. The method of example 6, wherein the indication of greater completeness as rate of change in overall variance decreases is jointly dependent on a history of variance in recent location readings.

Example 8. The method of any one of the preceding examples, wherein estimating the location based on the current location readings received comprises considering locations assigned to similar model location readings, wherein similarity between current and model location readings depends on distance between the current and the location readings in the measurement space.

Example 9. The method of any one of the preceding examples, wherein estimating the location comprises estimating based on model location readings and current location readings, and the more complete is the model, the larger is the distance between the current location readings and the model location readings used in the estimating.

Example 10. The method of example 8 or 9, wherein the distance is Euclidean.

Example 11. The method of any one of the preceding examples, comprising:
providing a first estimate and a second estimate of the location; and
estimating the location of the catheter based on the first estimate, the second estimate, and the completeness indicator.

Example 12. The method of example 11, wherein estimating the location comprises:
weighting the first estimate and the second estimate based on the completeness of the model indicated by the completeness indicator; and
averaging the first and second estimates according to the weights.

Example 13. The method of example 12, wherein at least one of the first estimate and the second estimate is based on an approximation approach, wherein the approximation approach comprises transforming the current location readings to locations using a transformation that approximates the transformation used to transform the model location readings to locations for generating the model.

Example 14. The method of example 12 or 13, wherein at least one of the first estimate and the second estimate is based on a nearest neighbor approach, wherein the nearest neighbor approach comprises determining the location based on locations assigned to model location readings similar to the current model readings, with similarity between given location readings being dependent on a distance between the given location readings in a measurement space and a nearest neighbor parameter.

Example 15. The method of example 14, wherein the first estimate is based on the nearest neighbor approach with a first nearest neighbor parameter, and the second estimate is based on the nearest neighbor approach with a second nearest neighbor parameter, different than the first nearest neighbor parameter.

Example 16. The method of example 11, wherein estimating the location comprises: considering locations assigned to model location readings that are similar to the current location readings, wherein in generating the first estimate, similarity between location readings depends on Euclidean distance between the location readings in a measurement space; and in generating the second estimate, similarity between location readings depends on geodesic distance between the location readings in the same measurement space.

Example 17. The method according to any one of examples 1 to 16, wherein estimating the location comprises selecting an estimation method based on the completeness indicator; and estimating the location using the selected estimation method.

Example 18. The method according to any one of examples 1 to 17, wherein estimating the location comprises using an estimation method that depends upon a value of a parameter, and setting the value of the parameter based on the completeness indicator.

Example 19. An off-line method of estimating a location of a catheter in a body part with respect to a model of the body part, wherein the model was generated based on model location readings received from the catheter in advance of carrying out the method, the method comprising:
receiving a completeness indicator indicative of a completeness the model had when current location readings were taken by the catheter when the catheter was in the location to be estimated,
receiving the current location readings taken by the catheter when the catheter was in the location to be estimated; and
estimating the location based on the received current location readings and the completeness indicator.

Example 20. The off-line method of example 19, wherein the completeness indicator is the time that lapsed from when the model generation has started and the time the current location readings were taken by the catheter.

Example 21. The off-line method of example 19, wherein the completeness indicator is based on variance in model location readings taken by the catheter before the current location readings were taken.

Example 22. The off-line method of any one of examples 19 to 21, wherein estimating the location based on the current location readings comprises considering locations assigned to similar model location readings during the generation of the model, wherein similarity between location readings depends on distance between location readings in a measurement space.

Example 23. The off-line method of any one of examples 19 to 22, wherein estimating the location comprises estimating based on model location readings, taken by the catheter before the catheter was in the location to be estimated, and current location readings, taken by the catheter when the catheter was in the location to be estimated, and the more complete is the model, the larger is the distance between the current location readings and the model location readings used in the estimating.

Example 24. The off-line method of example 22 or 23, wherein the distance is Euclidean.

Example 25. The off-line method of any one of examples 19 to 24, comprising:
providing a first estimate and a second estimate of the location;
weighting the first estimate and the second estimate based on a completeness of the model indicated by the completeness indicator; and averaging the first and second estimates according to the weights.

Example 26. The off-line method of example 25, wherein estimating the location comprises:
considering locations assigned to model location readings similar to the current location readings, wherein in generating the first estimate similarity between location readings depends on Euclidean distance between the location readings in a measurement space; and in generating the second estimate similarity between location readings depends on geodesic distance between the location readings in the same measurement space.

Example 27. The off-line method of any one of examples 19 to 26, wherein the body part is a heart chamber.

Example 28. A method of guiding navigation of a catheter within a body part, the method comprising:
showing an estimated location of the catheter with respect to the body part, on a model of the body part, generated based on location readings received from the catheter, and
updating the shown location as new location readings are received from the catheter,
wherein the location is estimated by:
   receiving completeness indicator, indicating a completeness of the model,
   receiving current location readings from the catheter when the catheter is in the location to be estimated, and
   estimating the location based on the current location readings received, and the received completeness indicator.

Example 29. The method of example 28, wherein the completeness indicator is based on the time that lapsed from when the model generation has started and the time the current location readings were received.

Example 30. The method of example 28, wherein the completeness indicator is based on variance in model location readings.

Example 31. The method of any one of examples 28 to 30, wherein estimating the location based on the current location readings comprises considering locations assigned to similar model location readings during the generation of the model, wherein similarity between location readings depends on distance between location readings in a measurement space.

Example 32. The method of any one of examples 28 to 31, wherein estimating the location comprises estimating based on model location readings, and current location readings, and the more complete is the model, the larger is the distance between the current location readings and the model location readings used in the estimating.

Example 33. The method of example 31 or 32, wherein the distance is Euclidean.

Example 34. The method of any one of examples 28 to 33, comprising:
providing a first estimate and a second estimate of the location; and
estimating the location of the catheter based on the first estimate, the second estimate, and the completeness indicator.

Example 35. The method of example 34, wherein estimating the location comprises weighting the first estimate and the second estimate based on the completeness of the model indicated by the completeness indicator; and averaging the first and second estimates according to the weights.

Example 36. The method of example 35, wherein the first estimate is based on an approximation approach and the second estimate is based on a nearest neighbor approach.

Example 37. The method of example 35, wherein the first estimate is based on a nearest neighbor approach with a first nearest neighbor parameter, and the second estimate is based on a nearest neighbor approach with a second nearest neighbor parameter, different than the first nearest neighbor parameter.

Example 38. The method of example 35, wherein estimating the location comprises:
considering locations assigned to model location readings during the generation of the model that are similar to the current location readings, wherein in generating the first estimate, similarity between location readings depends on Euclidean distance between the location readings in a measurement space; and in generating the second estimate, similarity between location readings depends on geodesic distance between the location readings in the same measurement space.

Example 39. The method of any one of examples 28 to 38, wherein the body part is a heart chamber.

Example 40. An apparatus for guiding navigation of a catheter within a body part, the apparatus comprising:
a processor; and
a display, configured to show an estimated location of the catheter with respect to a model of the body part responsive to instructions received from the processor,
wherein the processor is configured to:
instruct the display to show the estimated location; and
determine the location to be shown based on inputs comprising:
completeness indicator, indicating a completeness of the model; and current location readings received from the catheter when the catheter is in the location to be estimated.

Example 41. The apparatus of example 40, wherein the processor is further configured to generate the model from location readings received from the catheter.

Example 42. The apparatus of example 40 or example 41, wherein the processor is configured to determine the completeness indicator based on time that lapsed from when data receipt from the catheter in the body part started, and the time the location readings used for determining the location to be shown were received.

Example 43. The apparatus of example 40 or example 41, wherein the processor is configured to determine the completeness indicator based on variance in location readings received to generate the model.

Example 44. The apparatus of any one of examples 41 to 43, wherein the processor is configured to determine the location to be shown based on the location readings received from the catheter from the location to be shown, and locations assigned to similar location readings during the generation of the model, wherein similarity between location readings depends on distance between location readings in a measurement space.

Example 45. The apparatus of any one of examples 41 to 44, wherein the processor is configured to determine the location to be shown based on model location readings, received from the catheter before the catheter was in the location to be estimated, and current location readings, received from the catheter when the catheter is in the location to be estimated, and the more complete is the model, the larger is the distance between the current location readings and the model location readings used in the determination.

Example 46. The apparatus of example 44 or 45, wherein the distance is Euclidean.

Example 47. The apparatus of any one of examples 40 to 46, wherein the processor is configured to:
generate a first estimate and a second estimate of the location; and
determine the location to be shown based on the first estimate, the second estimate, and the completeness indicator.

Example 48. The apparatus of example 47, wherein determining the location to be shown comprises weighting the first estimate and the second estimate based on the completeness indicator; and averaging the first and second estimates according to the weights.

Example 49. The apparatus of example 48, wherein the first estimate is based on an approximation approach and the second estimate is based on a nearest neighbor approach.

Example 50. The apparatus of example 48, wherein the first estimate is based on a nearest neighbor approach with a first nearest neighbor parameter, and the second estimate is based on a nearest neighbor approach with a second nearest neighbor parameter, different than the first nearest neighbor parameter.

Example 51. The apparatus of example 48, wherein the processor is configured to determine the location by:
considering locations assigned to model location readings received during the generation of the model that are similar to the current location readings received when the catheter is in the location to be shown, wherein in generating the first estimate similarity between location readings depends on Euclidean distance between the location readings in a measurement space; and in generating the second estimate similarity between location readings depends on geodesic distance between the location readings in the same measurement space.

Example 52. The apparatus of any one of examples 40 to 51, wherein the body part is a heart chamber.

In some examples, a method of estimating a new location of a catheter in a body part of a body using a new location reading received from the catheter is provided, wherein transformation for transforming location readings to locations was generated based on previous location readings received from the catheter before the new location reading was received. The method comprises receiving the new location readings and estimating the new location of the catheter based on the new location reading and a similarity indicator. The similarity indicator is indicative of a similarity between the new location reading and the previous location readings. The new and previous location readings may each comprise a measurement of voltages generated by respective electric fields at an electrode disposed on the catheter, wherein the electric fields are mutually non-parallel and were generated by electrodes disposed in a fixed relationship to the body and each location reading may be part of a set of location readings received from corresponding electrodes disposed on the catheter. The transformation may be computed to preserve distances between locations of the electrodes on the catheter.

As is discussed in further detail below, in many use cases the model and transformation are both updated as new information becomes available, for example periodically at a frequency that allows in enough time to carry out the updates, typically at a lesser rate than that at which data is collected or catheter positions are calculated and optionally displayed. Therefore, the completeness indicator and similarity indicator may be linked as the model is in these examples eventually updated as new location readings become available. In other words, for a new location reading with a similarity indicator indicating little similarity with previously obtained readings used previously to calculate the transformation, the new location reading will be in a region with no or only a few previously obtained readings and hence the model will be incomplete in that region and have a corresponding completeness indicator. On the other hand, for a new location reading with a similarity indicator indicating greater similarity with previously obtained readings, the new reading will be in a region with previously obtained readings that may have been used to update the model, so that the completeness indicator indicates a complete model in that region.

The similarity indicator may be indicative of a distance between the new location reading and one or more of the previous locations in a space of location readings. Estimating the location of the catheter based on the similarity indicator may comprise selecting an estimation method based on the similarity indicator and estimating the location using the selected estimation method. The estimation method may be selected from among two or more of the group: estimating the location using the transformation; generating an updated transformation using the previous and new location readings, wherein the updated transformation is an approximation to the transformation generated based on the previous and new location readings and using the updated transformation to estimate the location; and estimating the new location using previous locations estimated for selected previous location readings, wherein the selected previous location readings have been selected based on the similarity indicator or wherein the selected previous location readings are one or more nearest neighbours of the new location reading in location reading space. Estimating the new location may be based on one or more previous locations estimated for respective selected previous location readings, wherein the selected previous location readings have been selected using the similarity indicator with or without selecting of an estimation method. Similarly, estimating the new location may be based on one or more previous locations estimated for selected previous location readings, wherein the selected previous location readings are nearest neighbours to the new location reading in a space of location readings.

In any of the above examples, the body part may be a heart chamber. The above examples may extend to being implemented by a computer-readable medium or media comprising instructions that, when run on a processor, implement the example and to a system comprising a processor and a memory storing instructions that, when run on the processor, implement the example.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing.

In the context of this document, a computer readable storage medium may be any tangible medium that can contain, or store a program for use by or in connection with an instruction execution system, apparatus, or device.

A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 is a flowchart of a method of estimating a location of a catheter in a body part, according to some embodiments of the present disclosure;

FIG. 2 is a flowchart of a method of estimating location of a catheter, according to some embodiments of the present disclosure;

FIG. 3 is a flowchart of a method of guiding navigation of a catheter within a body part, according to some embodiments of the present disclosure; and

FIG. 4 is a simplified block diagram of an apparatus for guiding navigation of a catheter within a body part, according to some embodiments of the present disclosure.

### DESCRIPTION OF SPECIFIC EMBODIMENTS OF THE INVENTION

The present invention, in some embodiments thereof, relates to the field of navigation of body cavities by intra-body probes, and more particularly, to reconstruction of body cavity shape from measurements by intra-body probes.

### Overview

In a previous patent application, WO 2018/130974, Applicants described methods of modelling a heart chamber based on location readings received from a catheter inside the heart chamber. This is also applicable to other organs or body parts that one may wish to model using measurements (readings) obtained using a sensing catheter such as an electrode catheter.

Herein, “location reading” is a term encompassing any reading that may be used for determining the location of the catheter. These are usually electrical readings (e.g., measurements of voltage), read by an electrode (or a plurality of electrodes) carried by the catheter. In some embodiments, location readings are obtained when some electrical fields are applied by intra-body electrodes (e.g., carried by the catheter), and/or by external electrodes (e.g., attached to the outer skin surface of the patient, whose body part is being catheterized). In some embodiments, the location readings include location readings indicative of the applied fields; for example, location readings measuring voltage and/or current at the internal and/or external electrodes that apply the fields.

According to some of the previously disclosed embodiments, the model is built during a catheterization procedure, and as the catheter probe visits more and more parts of the heart chamber, these parts are added to the model. So the model itself develops during the procedure. The model is a graphical representation of the modelled heart chamber, for example, a representation of the inner wall of the heart chamber, as viewed from within the heart chamber. The model may be considered “complete” when additional data indicative of additional probe visits is incorporated into the model, the model does not change its shape, at least not to an extent that such a change is evident to a viewer of the model, viewing the model on a display. For example, as the probe visits more sites near the inner wall of a heart chamber, the model of the heart chamber, showing this inner wall becomes more complete, until the entire wall is sampled, and receiving additional samples does not anymore significantly change the shape of the modeled wall. The completeness of the model can be, in some embodiments, local. For example, as long as the probe probes one region of the heart chamber wall, the model becomes more and more complete in this region; however, if the probe starts probing a new region of the wall, the model is far from completeness at the new region, and as probing of the new region continues, the model approaches completeness in the new region.

The present disclosure relates to a discovery by the Applicant that when new location readings are received from the catheter, there is a potential advantage to incorporating them into the model with reference to a metric correlated to completeness of the model. For convenience, in the present specification and claims, reference may be made to “model location readings”, which are location readings used for generating the model, and current location readings, which are the new location readings. It is noted, however, that the same location reading may be a current location reading when it is received, and then “become” a model location reading, when it is used to further develop the model. For example, in embodiments where measurements are transformed to locations using a transformation generated based on all the available measurements, when the model is complete, it may be efficient to transform the new (current) measurements to location readings using the same transformation used for generating the model from previous (model) location readings. However, when the model is only in its initial stages, and the new data may cause the model itself to change, it may be prudent to use other transformations. For example, it may be prudent to use the new location reading as a model location reading, and update the model to incorporate it, and only then to treat the location reading as a current reading, and transform it to a location based on the updated model. However, such a procedure may be very expensive computationally. Therefore, the inventor found a need to provide more reliable results than using a young (i.e., incomplete), not updated model, at less computational cost than fully updating the model. One solution to that problem, is to find some old (model) location readings similar to the new (current) location readings, and transform the new location readings to locations similar to those to which the old location readings were transformed. This was found to provide more reliable results than using an un-updated incomplete model, and less computationally expensive than updating the incomplete model before using it to transform the new location reading to a location.

Put in terms of the transformation used for transforming location readings to locations, once new measurements don't change significantly the transformation, there is no need to update the transformation in order to transform the new measurements to new location readings. Instead, the previously calculated transformation can be applied without update to new measurements, in some cases until a next update of the transformation. The next update may be for example after a pre-determined time, when a certain number of location readings have been collected, or some other condition, for example the collection of a certain number of location readings that add new information or have a difference above a threshold from the samples used to calculate the previous transformation. However, when the previously calculated transformation is not sufficiently accurate, for example if the new location reading has a difference from previously collected location readings that is above a threshold (or, similarly, a similarity measure below a threshold), an alternative approach that is less computationally intense than a full update of the transformation but yields more accurate results than using the non-updated transformation may be used. For example, one or more nearest-neighbour location readings may be used together with their previously estimated location to estimate a new location for the new location reading. In another example, the transformation may be updated using an approximate, less computationally intense method using the new location reading and the approximately updated transformation used to estimate the new location from the new location reading. In some cases, it may be sufficient to find some similar measurements that have been taken before, and transform the new measurement to a location based on the locations to which the similar measurements have been transformed rather than using the transformation, even if the transformation is sufficiently accurate. For example, if the model is complete, it may be easier to allocate to the new reading a location in the vicinity of locations allocated to similar readings, then updating the full transformation.

It will be appreciated that in most use cases, both the model and the transformation will be updated as new location readings arrive, for example periodically, after collecting a certain number of location readings or a certain number of location readings sufficiently different from previously used readings (see above for example). Although the model and transformation need not be updated on the same schedule or at the same time, this means that generally an indication of completeness of the model will be indicative on an accuracy of the transformation in the relevant region and vice versa.

Accordingly, a first aspect of the present disclosure is a method of estimating a location of a catheter inside a body part (e.g., in a heart chamber) with respect to a model of the body part, wherein the model was generated based on location readings received from the catheter.

The method includes, in some embodiments: receiving location readings from the catheter when the catheter is in the location to be estimated; and estimating the location based on an indicator to a completeness of the model, also referred to herein as “completeness indicator”.

Embodiments optionally use one or more of a plurality of indicators to the completeness of the model. For example, a completeness indicator optionally comprises duration elapsed from when the model generation has started and the time the location readings were received from the location to be determined. Heuristically, the longer the time, the more complete is the model. This indicator has some potential disadvantages. One is that at some point, the model is quite complete, so that further location readings don't add to its completeness. Therefore, many different values of the time indicator could potentially indicate effectively the same level of completeness. Another potential disadvantage is that sometimes a physician starts roving with the catheter in the body part, pauses in one spot for some dwelling time and only then continues the roving. During this dwelling time, the model is not developing, while the time keeps running. Despite these potential disadvantages, the time indicator was found to be easy to implement, and to provide satisfactory results, although better indicators may be developed. The present disclosure is relevant to any such indicator, and not limited to the indicators described herein.

In some embodiments, an indicator to the completeness of the model is based on variance in location readings received to generate the model. When all the location readings are from the same neighborhood, the variance in location readings is relatively small. If a new region is being visited by the catheter, after an old region is visited extensively, the variance may grow abruptly. Thus, a steady variance of the location readings in the region containing the location to be estimated may be indicative of a complete model in that region, while an increasing variance may be indicative of an incomplete model in that region. In some embodiments, presumed completeness of the model increases as overall variance increases, as an indication of how many different regions have been mapped. In some embodiments, a variance metric is coupled to another metric, optionally time (e.g., completeness optionally requires both passage of time and establishment of a certain level of variance), and/or another measure of variance. For example, a completeness indicator may use total variance as a measure of how much of a structure has been mapped, and recent variance (e.g., variance within the last 5-30 seconds) as a measure of activity. A lack of total variance increase during non-movement of the catheter (e.g., a recent history of readings is also low in variance) is optionally disregarded as a potential indicator of model completeness.

In some embodiments, the estimation of the location of a catheter is based on location readings received while the catheter was at that location, for example as follows.
Identify similar location readings received beforehand (“old similar location readings”).
Estimate the current position based on the positions estimated for the old similar location readings.

In some embodiments, what location readings are considered “similar”, optionally changes as the model's completeness indicator shows greater completion.

For purposes of explanation, the received measurements are optionally considered to represent “points” in a “measurement space”. For example, if there are three electromagnetic fields at the heart chamber during the procedure, and the catheter measures all three of them at each point in space, where the catheter is, then each such triplet of measurements is a point in measurement space. If there are more or less than three fields, and/or another measurement parameter (e.g., a measurement of local dielectric properties), the measurement space may have correspondingly more or less dimensions.

One way to define “similarity” between measurements is by their distance from each other in the measurement space. Accordingly, estimating the location of the catheter based on the location readings received from that location may include considering locations assigned to similar location readings during the generation of the model, wherein similarity between location readings depends on distance between location readings in the measurement space (“nearest neighbor approach”). It should be understood that a calculation of distance in measurement space is distinct from a physical distance between two locations; although potentially two readings which are close in measurement space are correspondingly obtained from nearby locations.

For example, one may find the nearest neighbors of the new location reading in the measurement space. The nearest neighbors may be defined, for example, as the 4, 6, or any other predetermined number of location readings that are closest to the new location reading. Optionally, the distance is measured by Euclidean distance. Additionally or alternatively, the nearest neighbors may be defined as all the neighbors that are within a predetermined radius from the new location reading. The predetermined radius and/or number of location readings N, may be referred to as a “nearest neighbor parameter”. Then, one can see what location was estimated for each of the old location readings (“old locations”), and determine the location of the catheter when the catheter takes the new location reading as a weighted average of the old locations. The weighting may be such that the closer an old location reading is to the new location reading, the higher is its weight in the average.

It was found that this method potentially provides satisfactory results when the model is far from completeness: for example, in the beginning of a catheterization process, or when the catheter starts visiting a region not visited before in the process (e.g., enters a vein not entered before).

When the model is more complete, another way of estimating a location for the new location readings is potentially preferable. For example, the more complete is the model, it is useful to rely on old location readings that are more distanced from the new location readings. This can potentially be, for example, because pathways and/or gradients between readings from more widely separated points have become organized and/or completed in the model to a sufficient degree that they begin to be useful as a basis for location finding. Thus, in some embodiments, when the model is far from completeness, Euclidean distances are used to estimate similarity between new and old measurements. When the model is closer to completeness, geodesic distance may be used to estimate the similarity between the new and old measurements. Geodesic distances are further discussed below.

It was also found that it is potentially useful to generate a plurality of estimates for the new locations, and average the estimates with weights that depend on the completeness of the model. For example, a first estimation of the new location may be obtained using Euclidean distances, and a second location may be obtained using geodesic distances. Each estimation may be associated with a weight depending on the completeness indicator (e.g., higher completeness may be associated with larger weight for the geodesic distance), and the weighted estimations may be averaged according to their weights to obtain an improved estimation of the new location.

A geodesic distance, which may also be referred to as a “natural” distance. In some embodiments, for example, the measurements may form a measurement cloud (in some measurement vector space, for example), and the spatial positions to which the measurements are transformed may form a position cloud, also referred to herein as R-cloud. The measurement cloud is also referred to herein as V-cloud. In some embodiments, a natural (geodesic) distance between two measurements may be defined as the length of the shortest path that goes between the two measurements only through the measurement cloud. Similarly, a natural distance between two spatial positions may be defined as the length of the shortest path that goes between the two spatial positions only through the position cloud.

In a previous application, Applicants described a transformation that transforms location readings to locations. This transformation is based on the location readings, so at least as long as the model is not complete, when new location readings arrive, the transformation has to be updated before it is useful for transforming the new location readings to new locations. When the model is complete, there isn't much to update, and the old transformation, generated before the new location readings arrived, may be useful for transforming the new location readings. However, at low or intermediate degrees of completeness, it is possible to approximate the updated transformation (“the approximation approach”). Such an approximation may be less computationally expensive than a full update of the model, and provide more accurate locations than provided by the nearest neighbor approximation.

The approximated transformation is then optionally used to transform the new location readings to new locations, and these new locations are averaged with new locations found according to the nearest neighbors (defined in Euclidean distances). The average is weighted according to the indicator of the completeness of the model.

Example of a transformation based on measurements, and an approximation thereto

To explain the approximation that may be used for an exemplary transformation, one way of generating a transformation based on measurements is described herein, and then an approximation thereto is provided. However, the present invention is not limited for use with this specific transformation and/or approximation.

In some embodiments, reconstruction (and/or in particular transformation generation) of a body cavity shape and/or navigation in a body cavity may be obtained by first assuming local spatial position constraints which are consistent with the physical conditions applying to individual sets of measurements (like the known relative distance of measuring sensors at the time the measurements were taken). In some embodiments, this assumption is combined with use of a multidimensional scaling (MDS) algorithm. MDS algorithms refer to a class of algorithms wherein objects (in some embodiments, measurements of voltage) are placed in an N-dimensional space (e.g., as described herein, the three dimensional space of a body cavity) so that between-object distances are preserved as well as possible (given all other, potentially competing, constraints). In some embodiments, the geometrical configuration of sensors on an intra-body probe provides the between-object distances, allowing an MDS approach to be used for reconstruction of a body part. In some embodiments, the configuration is fixed (e.g., a rigid catheter section). In other embodiments, the configuration may be flexible (e.g., a flexible probe section or multiple probes), however, there may still be useful constraints on the relative positions of probe sections, such as possible distances between sensors due to probe flexibility and deformability limitations and/or other properties. In addition, estimations of geometrical properties of the probe (or probes) and interrelations between sensors carried thereon may be used; for example, probe position values and/or sensor position values provided by position sensors and/or restrictions on movement provided by nearby structure and/or based on possible speed of movement of parts of the probe. It is noted that many of these constraints are local (e.g., relate to volumes with a diameter of less than 50%, 20%, 10% or intermediate percentages of a largest dimension of the reconstructed shape). In some embodiments, more global constraints are used, for example, on an overall shape of the transformation, on a uniformity of the transformation (e.g., as compared to a generic transformation based on generally expected behavior of electric fields in the body) and/or based on expected distances between closest simultaneous measurements.

In some embodiments of the invention, several sets of measurements x are obtained in X; each set x being made up of a plurality of measurements xᵢ, xⱼ, . . . measured simultaneously by different sensors i,j on a same probe; and with distances (e.g., or other geometrical constraints) between at least some of the sensors being known or estimated (e.g., including bounded), so that the distances can be used as a constraint. Moreover, in some embodiments, more than one measurement is made from each sensor (e.g., measurements of different electrical fields, e.g., of fields having different frequencies), so that the set of measurements in total includes, e.g., xᵢ_{_{1,2 . . .}} , xⱼ_{_{1,2 . . .}} , . . . . It is noted that these constraints are optionally recalculated as part of the reconstruction.

Measurements in a set are optionally taken substantially simultaneously, i.e., while the probe remains in substantially the same position. Moreover, in some embodiments, the different measurement locations on the probe optionally have known spatial relationships to one another, which comprise, in some embodiments, local spatial position constraints. Optionally, reconstruction of the body cavity shape is guided based on these known spatial relationships. For example, in some embodiments, a transform function T(x) on a each member of group of measurements X comprising the set of measurements x may be calculated such that |T(Xᵢ)−T(Xⱼ)|≈dᵢⱼ; dᵢⱼ being the distance between electrode; and electrodeⱼ.

For example, in some embodiments, the electrodes are each at a known distance and/or angle from one another due to a fixed geometry of the intra-body probe to which they are mounted. Alternatively, in some embodiments, electrodes are in variable relative positions, and the variation accounted for based on information such as parameters of deployment (e.g., how expanded a basket-shaped intra-body probe is at a moment of measurement), and/or on further measurements (for example, of force as an indication of probe deformation, inter-electrode conductance as an indication of inter-electrode distance, etc.). Optionally, additional constraints on the relative orientation of the measurement locations are also used. Such constraints are optionally known, for example, from geometrical/anatomical constraints on the procedure itself.

Optionally, measurements in each set are substantially simultaneous. Herein, “substantially simultaneous” should be understood to mean that the measurements of each set may be obtained:
Actually simultaneously (i.e., with partially or wholly overlapping measurement periods),
Close enough in time that motions of the intra-body probe during acquisition of the set can be neglected, and/or
Close enough in time that skew due to small movements during sampling of a set of measurements can be dependably factored out and/or adjusted for if necessary (e.g., by use of time-weighted averaging of time-adjacent samples).

Optionally, a collection of measurements is considered as a set of measurements mutually constrained in relative position (e.g., fixed at particular relative distances and/or relative angles, at variable but known distances or angles, for example by use of an encoder, etc.), without a requirement for substantial simultaneity of measurement. For example, multiple measurements at multiple times from an intra-body probe are optionally taken while a portion of the intra-body probe remains anchored at one or more regions. Relative movements of other intra-body probe portions, assuming they are known (by use of a movement encoder, for example) can then be applied to determine a relative position constraint. These measurements are optionally related to one another through use of the fixed anchor and the known bending parameters to provide calibration. It can be understood from this, and it should be understood to apply generally, that a measurement (also known as a “measurement sample”) optionally is treated as a member of a plurality of “sets” of measurements, where members of each set may be related to one another through application of different mutual position constraints. “Readings”, in some embodiments, comprises such measurement sets.

For simplicity, and for purposes of description herein, sets of simultaneous measurements from corresponding electrodes of a fixed-shape probe are often used in examples. However, it should be understood that other configurations of sensors, and/or other methods of obtaining a spatially calibrated “ruler” to constrain distances between them are optionally used in some embodiments of the present invention. In some embodiments, the constrained distances may be used to ensure that the target shape is reconstructed so that the distance between the electrodes (e.g., in mm) is kept approximately the same all around the reconstructed shape, even if the difference between their readings (e.g., in mV) changes substantially from one place to another. For example, in some embodiments, the length of the catheter is reconstructed to be the same within +15% even though the voltage gradient between the same electrodes changes by a factor of 10 or more.

Herein, voltages measured substantially simultaneously by two electrodes separated from each other by a fixed distance (e.g., because they are fixed to a rigid probe portion), may be referred to as sister measurements; the locations assigned to such measurements may be referred to as sister locations; and the distances between sister locations may be referred to as sister distances. The fixed distance itself may be referred to as a desired sister distance.

In some embodiments, a transform function to be found is defined as comprising two terms: one which gives a roughly scaled transformation of V-cloud measurements into an R-cloud, and a second which applies displacements to that roughly scaled R-cloud. The second term potentially helps overcome at least some of the electrical field non-linearities and/or non-orthogonality which may exist in the roughly-scaled transformation.

The rough-scaling term of the displacement approach of some embodiments of the invention can be understood, for example, by envisaging each measurement set x of the measurements X to be first “copied” from a coordinate system in a measurement space, wherein each of the measurement space axes is, e.g., an axis of measurement values for one of a respective plurality of crossing electrical fields; to a coordinate system in a physical space, wherein different positions along the axes represent different locations in physical space. This copying may be carried out with a different scale along each axis; for example: a voltage difference of 1 mV measured along a horizontal axis in the measurement space may correspond to a distance of 3 mm along the horizontal axis of the physical space, and a voltage of 1 mV measured along a vertical axis in the measurement space, may correspond to a distance of 2 mm in the physical space. In notation form, the voltage points X may be envisaged to be first “copied” to initial location points Y, e.g., by a scaling transformation Y=diag(a)X, where a is, in some embodiments, a vector comprising scaling coefficients a=(aₓ, a_{y}, a_{z}), with units of distance/measurement (e.g., mm/mV). diag(a) indicates the matrix diagonalizing vector a. With the addition of a displacement term W, the initial location points diag(a)X are displaced by displacement W to have the proper local scaling (i.e., to make sister distances in Y optimally correspond to the known distances between the sensors). It is noted that while the axes in the physical space may be orthogonal, this does not limit the method to embodiments where the fields themselves are orthogonal to each other, or even close to orthogonality (e.g., the axes may be, for example, 20 degrees or more off axis, for example).

The axes in the physical space are provided as a convenient means for describing locations in space, and the transformation from the measurements to the positions by the rough-scaling term is arbitrary. Still, the more orthogonal are the fields, the less arbitrary is this transformation, and the computational effort required to find the optimal transformation may be smaller. In some embodiments of the invention, the rough-scaling term is mainly used for transforming the data from units of voltage (or other measurement) to units of length. In addition, if the data imply a need to stretch the reconstruction along some direction, the rough-scaling term can allow doing so using a smaller number of actions than would be required if only W was available for applying such stretching (e.g., in case the rough-scaling term was predetermined to be the same for all the fields.

The displacement term W can be decomposed in different ways in order to guide the search for the individual displacements that make it up. In some embodiments, accordingly, the displacement W is expressed as a multiplication of two matrices: W=UW′, with U being a representation of X in a coordinate system “natural” to X, and W′ being a matrix of coefficients (displacement coefficients) which give the magnitude of displacements applied within the same “natural” coordinate system, also referred to herein as a coordinate system that preserves the “intrinsic geometry” of X.

This intrinsic geometry, in some embodiments, is defined as comprising a set of linearly independent features (referred to as characteristic vectors or eigenvectors v of a similarity matrix, reflecting similarity between sampled measurements) which “sum up” (after individual scaling of the eigenvectors v, each by its eigenvalue) to produce an equivalent representation of X.

Decomposition of X into eigenvectors, in some embodiments, has the effect of separating features according to their spatial frequencies. This property is optionally used in relation to maintaining spatial coherence, for example as discussed hereinbelow.

In some embodiments, a kernel K is defined as a matrix that expresses a measure of the distances between each pair of measurements:

$${K}_{i , j} = K ( {x}_{i} , {x}_{j} ) = {e}^{\frac{- {‖ {x}_{i} - {x}_{j} ‖}^{2}}{2 {σ}^{2}}}$$

This form of a kernel is optionally referred to as a radial basis function kernel, and is an example of a similarity matrix. The sigma parameter is a free variable, which optionally is set to be about 0.1. Optionally, the kernel K is normalized to a normalized kernel \tilde{K}, for example by one of:

$$S = diag {\sum }_{j} {K}_{i , j} \tilde{K} = \frac{K}{S}$$

$$or$$

$${S}_{i} = diag {\sum }_{j} {K}_{i , j} {S}_{j} = diag {\sum }_{i} {K}_{i , j} \tilde{K} = \frac{K}{\sqrt{{S}_{i} {S}_{j}}}$$

$$or$$

$$S = diag ( K \cdot n ) \tilde{K} = {S}^{- 1 / 2} {KS}^{- 1 / 2} wherin n = [ \begin{matrix}1 \\ 1 \\ … \\ 1\end{matrix} ]$$

The normalized kernel \tilde{K} is decomposed to find U, for example, using the Graph Laplacian, such that for the k most significant eigenvectors u:

The eigenvector matrix U is: U=[u₁, . . . uₖ]
The eigenvalue matrix V is: V=diag([λ₁, . . . λₖ])
And the decomposition satisfies: \tilde{K}u=λu

Putting the terms just described together results in an X (measurement cloud) to Y (position cloud) transformation which may be expressed by the equation Y=diag(a)X+UW′.

The model is a graphical representation of the position cloud, for example, it may be an outer shell of the position cloud, or a mesh defining it. Finding and outer shell of a point-cloud is well known in the art, and may be accomplished, for example, using a pivoting ball algorithm.

Each set of a and W′ provides a configuration that gives a generally different transformation of X to Y. To find the transformation that provides a best fit between sister distances and the desired sister distances (e.g., known distances between the sensors on the probe), a penalty may be associated with each deviation of the sister distances from the known distances, and this penalty minimized by known minimization procedures adjusting (the components of) a and W′. Other penalties described herein are also optionally applied, e.g., by addition to the penalty on the difference between sister locations and known distances between sensors on the probe. A choice of a and W′ with a minimal penalty result gives, from the point of view of the algorithm and its particular cost function, the “correct” Y from the given X.

Using standard techniques, the decomposition calculation that determines U, explained above, is computationally expensive. When new measurement data is acquired, it is a potential advantage to avoid the necessity to perform the entire decomposition each time, for example, in order to allow faster updating of the reconstruction results.

In some embodiments, recalculation of the decomposition is performed based on the following, given new measurements \hat{X} after already having obtained a component decomposition U using older measurements X:

The new kernel \hat{K} is:

$$\hat{K} = K ( \hat{{x}_{i}} , {x}_{j} )$$

The normalization matrix Ŝ is: diag (\hat{K} [symbol] )
The new decomposition Û is: Û=Ŝ^{-1/2}\hat{K}S^{-1/2}UV⁻¹
And the equation for which a and W′ are to be optimized is: Ŷ=\hat{X}diag(a)+ÛW′

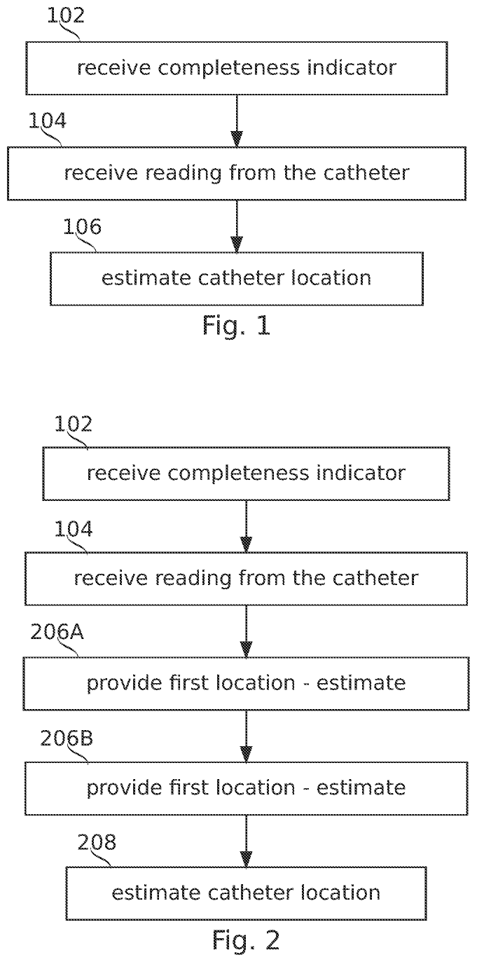

In summary, rather than calculating the full decomposition U and adjusting a and W′ using an error minimization technique as described above, the approximate method uses the previously calculated a and W′, calculated on the previously obtained location readings, and an approximate decomposition Û calculated on the set of measurements \hat{X} including the previous location readings and the new location reading(s) that have not yet been used for a full update of the transformation. By avoiding the optimization of a and W′ and using an “online” approximate update for U, less computation is required to calculate the approximately updated transformation than would be required for a full update. Reference is now made to FIG. 1, which is a schematic flowchart of a method of estimating a location of a catheter in a body part (e.g., heart chamber) with respect to a model of the heart chamber, generated based on location readings received from the catheter, and an indicator to a completeness of the model. The method may be carried out after some initial model has already been generated. For example, the received location readings may be indicative of location of the catheter inside the body part, and the indicated location is to be shown on a model of the body part.

In box 102, the completeness indicator is received.

In box 104, location readings from the catheter are received.

In box 106, the location of the catheter is estimated based on the location readings received, and the indicator to the completeness of the model. For example, the position of the catheter may be estimated in two different ways to provide two estimates, and the two estimates may be weight-averaged by weights determined based on the completeness indicator. The estimated location is the location the catheter is estimated to have when the location readings received in 104 are taken.

It is noted that the method itself may be carried out online (i.e., when the catheter is in the estimated location) or off-line (e.g., in post-hoc analysis of data gathered during a catheterization procedure). Either way, professional medical expertise is not required for carrying out the method, and the method does not entail any risk to the patient, as it has no influence on the interaction between the catheter and the patient's body.

Here, and in other places in the present disclosure and claims, where estimation of a location of a catheter is mentioned, what is being estimated in fact is the location of electrode(s) carried by the catheter. The electrodes are usually carried at a distal end of the catheter, so the location estimated may be the location of the distal end of the catheter, but this is not necessarily the case, as electrodes may be carried out also by other parts of the catheter. In this disclosure and claims, the location of the catheter is treated as if it was the same as locations of one or more electrodes carried by the catheter. Similarly, the location readings received from the catheter are made by the electrodes carried by the catheter.

The received location readings are usually of voltages, but in some embodiments, location readings of currents and/or impedances may also be similarly used for estimating the location of the catheter.

In some embodiments, the completeness indicator of the model uses the time that lapsed from when the model generation has started (i.e., before the method began) and the time the location readings were taken at the location to be determined. Optionally, “completeness” is a nonlinear function of time (e.g., asymptotic), but it will typically be stable or increasing, unless, e.g., there is some reason (such as a change in the measuring system's configuration) to delete readings and/or restart mapping. When the method is carried out online, the time the location readings were taken is practically the same as the time at which the location readings were received.

Additionally or alternatively, the completeness indicator of the model, in some embodiments, uses variance in location readings received to generate the model. For example, in the beginning of the modelling process, every location reading is new, and enlarges the variance in the location readings. After some time, the location readings come from within a certain heart chamber or a certain portion of the heart chamber, for example from a vicinity of a landmark (e.g., mitral valve, pulmonary vein ostium, etc.) that has to be treated or observed carefully. In this stage, location readings are close to each other, and their contribution to the variance decreases, so the variance is stabilized on some value. Thus, the completeness indicator may be a function of the variance, e.g., may be equal to the variance, an inverse of the variance, or any other function. Preferably, the magnitude of the function result increases as the model is more complete.

In some embodiments, estimating the location based on the location readings received comprises considering locations assigned to similar location readings during the generation of the model. Such location readings may be referred to herein as “old location readings”, or “similar old location readings”, as the case may be. For example, associated with the similar old location readings may be averaged to obtain an estimation for the location of the catheter when the new location readings were taken.

In some embodiments, the old location readings may be saved in association with the old locations attributed to them, and old location readings similar to the new ones are searched among the saved old location readings. Similarity between location readings may be defined, for example, based on a distance between the location readings in the measurement space, so that the larger the distance (in measurement space) between location readings, the smaller is the similarity between them. In one example, location readings are considered similar to each other if the distance between them is smaller than a threshold. In another embodiment, a new location reading is considered similar to an old location reading if there is no other old location reading that is closer to the new one. In some embodiments, the N closest neighbors among the old location readings may be considered similar to the new location reading, with N being a whole number (nearest neighbor parameter), for example, 4, 5, 6, 7, 12, 16, etc. In some embodiments, when the model is young, the transformation is reliable mainly locally, and considering faraway neighbors might reduce the accuracy of the nearest neighbor approach. As the model completes, the transformation may become reliable on larger scales, and therefore further neighbors may add to the accuracy of the nearest neighbor approximation. Accordingly, in some embodiments, the more complete is the model (i.e., the higher completeness indicated by the indicator), more old location readings are considered similar to a new location reading, for example, N is larger. In another example, the threshold distance, under which location readings are considered similar to each other, is larger as the indicated completeness is higher.

In some embodiments, the averaging of old similar locations is weighted, so that different old locations may have different weight in the average. For example, the old locations may be weighted by an inverse (or any other monotonically decreasing function) of their distance from the new location, so the higher is the distance, the smaller is the weight of the old location.

In some embodiments, the distance used for determining similarity between measurements may be the Euclidean distance. For example, if location of a catheter is based on a location reading received from electrodes sensing three different electrical fields (e.g., having three distinct frequencies), the measurements may be three-dimensional. The location readings of one of the electrodes may be, for example, (V_{new}_{₁}, V_{new}_{₂}V_{new}_{₃}). An old location reading may be, for example (V_{old}_{₁}, V_{old}_{₂}V_{old}_{₃}), and the distance between the two points may be defined as

\sqrt{(V_{old}_{₁}−V_{new}_{₁})²+(V_{old}_{₂}−V_{new}_{₂})²+(V_{old}_{₃}−V_{new}_{₃})²}

In some embodiments, the distance between measurements may be defined by another kind of distance, for example geodesic distance. The geodesic distance between an old and a new location may be the shortest path between two locations that is confined to the model (e.g., always is within a certain maximum distance from a nearest location defined in the model, and/or locally oriented toward a nearest location defined in the model). These paths are potentially more meaningful in a more complete model, e.g., since in a more complete model they are less likely to be interrupted by gaps in the data which would potentially interrupt and/or distort the geodesic paths artifactually. If the model is based on a measurement cloud, transformed into a position cloud, the geodesic distance between measurements (e.g. location readings) may be the shortest path between two measurements in the measurements space, which is confined to the measurement cloud.

FIG. 2 is a flowchart of a method of estimating location of a catheter according to some embodiments of the invention.

In box 102, the completeness indicator is received.

In box 104, location readings from the catheter are received.

In box 206A, a first estimation of the location of the catheter is provided based on the location readings received.

In step 206B, a second estimation of the location of the catheter is provided, using a different estimation method, also based on the location readings received.

In step 208, the location of the catheter is estimated based on the first estimation, the second estimation, and the completeness indicator.

Boxes 102 and 104 are substantially the same as the corresponding boxes in FIG. 1.

The first and second estimations may be according to any one of the estimation methods described above. In some embodiments, weights are provided to the different estimations based on the completeness indicator, and the estimations are averaged according to the weights. For example, in some embodiments, a first of the estimation methods is judged potentially more effective than the other when the model is complete, and the other estimation method is judged potentially more effective than the first, when the model is far from completion. In such a case, the weight associated with the estimation is higher as the model is closer to completeness, and lower as the model is far from completeness. The weight associated with the second estimation may go the other way around, that is, be higher as the model is far from completeness, and lower as the model is close to completeness.

In some embodiments, one estimate is based on a nearest neighbor approach, and the other on an approximation approach. Optionally, the nearest neighbor parameter may also be determined based on the completeness indicator.

More particularly: a neighbor similarity-type location estimation may be preferred with a higher weight when the model completeness is low, potentially, e.g., because this estimation has low reliance on features of an overall structure which may be (at this stage) prone to artifacts due to restricted sampling area. For example, two visited areas linked by a path crossed with the probe moved from one to the other may give the model the overall appearance of a two-lobed “barbell”, even though the true structure is more globular in shape. This could introduce distortions where geodesic paths are artifactually forced, as a result of limited data availability, to pass through the region connecting the lobes.

Contrariwise, a geodesic path-type location estimation may be preferred with a higher weight when the model completeness is high. Once there are sufficient readings to allow geodesic paths through the model to assume a shape approaching their “true” shortest-path shape, a geodesic path provides a potential advantage by helping to maintain overall physical plausibility and continuity of the model. For example, when the readings are of parameters which continuously vary in space, a geodesic path-preserving transformation of readings to locations helps to ensure that this continuity is maintained in the model as well.

In some embodiments, the estimates are according to the nearest neighbor approach, but use different nearest neighbor parameter.

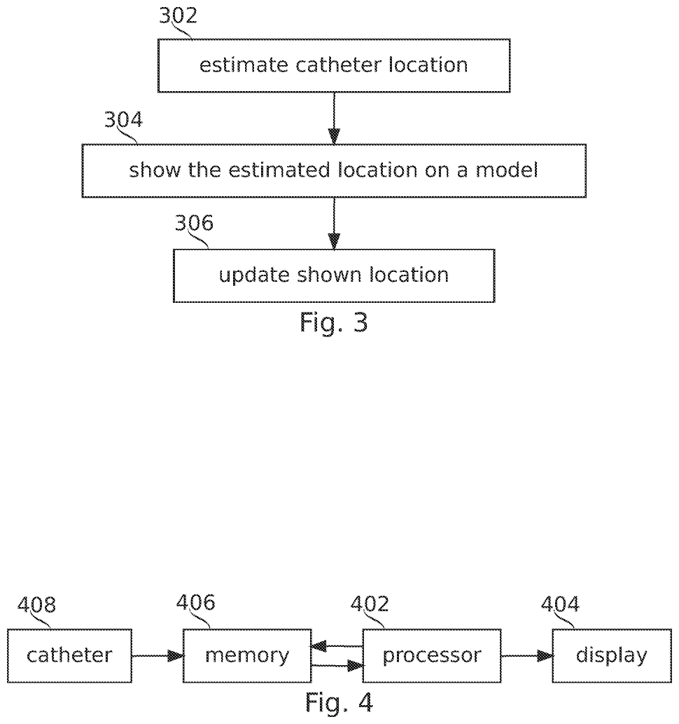

FIG. 3 is a flowchart of a method of guiding navigation of a catheter within a body part. In box 302 a location of the catheter with respect to the body part is estimated as described herein, for example, as in the method of FIG. 1 or the method of FIG. 2.

In box 304, the estimated location of the catheter with respect to the body part is shown on a model of the body part which was generated based on location readings received from the catheter. By seeing the estimated location on the model, an operator carrying out the catheter navigation can tell where the catheter is with respect of the anatomy of the patient, and navigate the catheter towards its goal in the body.

In box 306, the shown location is updated as new location readings are received from the catheter, so that movements of the catheter inside the body part are reflected on the shown model. This may be carried out by estimating the locations considering new location readings, as these are received from the catheter.

FIG. 4 is a simplified block diagram of an apparatus for guiding navigation of a catheter 408 within a body part (not shown). The apparatus includes processor 402 and display 404.

Processor 402 is configured to retrieve data from a digital memory 406. Digital memory 406 may store data received from catheter 408 during a catheterization procedure. In some embodiments, the processor is configured to retrieve the data from the digital memory after the catheterization has been finished, for example, for replaying the catheter movements for training purposes. Additionally or alternatively, the processor may be configured to retrieve the data from the memory in real time, in order to guide the operator in navigating the catheter during the catheterization procedure. Processor 402 is configured to determine the location of the catheter based on the data received from the catheter and a completeness indicator. The completeness indicator may be determined by the processor, and/or read from digital memory 406. For example, the data stored in digital memory 406 may include, further to the data received from the catheter, time stamps, indicating when each piece of data (e.g., data entry or a group of data entries) is received from the catheter. The processor may be configured to determine the completeness indicator based on the time stamps, for example, as described above. Additionally or alternatively, the processor may be configured to evaluate the variance in the data received from the catheter, e.g., from the beginning of the catheterization process to the receipt of the piece of data at hand, and determine the completeness indicator based on the variance.

Processor 402 is further configured to instruct display 404 to show the estimated location, estimated based on the location data received from the catheter and the completeness indicator determined by the processor.

Display 404 is configured to show the estimated location of the catheter with respect to a model of the body part responsive to instructions received from processor 402.

As mentioned above, processor 402 may be configured to cause the display to show the location of the catheter with respect to a model of a body part. In some embodiments, this model is generated by processor 402 from the location readings. Accordingly, in come embodiments, the model of the body part and the location of the catheter with respect to this model are both generated based on same location readings, although the model can be generated based on location readings received when the catheter was at different places, while the location of the catheter is based on location readings received in real time (except for the reliance on the completeness parameter, which may be determined based on all the location readings used for generating the model, or on some of those location readings).

Processor 402 may be configured to determine the location to be shown (that is, the location of the catheter at the moment being shown) in any one of the methods described above.

For example, processor 402 may be configured to determine two estimates of the location to be shown, and combine them (e.g., by averaging) according to the completeness indicator to provide a final estimate of the location.

### Example of Catheterization Guiding

To illustrate how embodiments of the present disclosure may be used in a catheterization procedure, consider an atrial ablation procedure, where the catheter has just entered the left atrium.

The catheter sends location data to the processor. For example, in some embodiments, three pairs of patch electrodes may be attached to the skin of the patient, and via each electrode pair an AC signal of a different frequency is transmitted. The AC signals may originate in a field generator. The catheter includes a plurality of electrodes, spaced apart from each other by known distances, and each electrode reads each of the AC signals, and sends these readings to the processor as location readings. The readings may be considered location readings, because at each location within the body, and particularly within the left atrium, the readings will be different.

The readings are collected, for example, at a rate of 100 readings per second. For example, if there are four electrodes, each reading three fields, the location data may be generated and received at a rate of 1200 data samples per second. When enough data is collected to generate a model (e.g., some 5 seconds after entering the left atrium), the location readings may be transformed to locations (e.g., from voltage readings to estimated locations in real space). Starting from the next time data arrives, e.g., 10 milliseconds after the model is generated, one or more further location readings are received.

The model, however, is optionally not immediately altered. The location readings are transformed to locations, and these locations are attributed as the catheter position at the time the data was received. The transformation used, in some embodiments, is an approximation (providing an approximation approach) simpler than the one used for generating the model, potentially allowing it to provide its output more quickly, e.g., to allow following the catheter in real time.

This simpler transformation may be, for example, based on the nearest neighbor approach and/or on the approximation approach. In view of the preliminary form of the model (indicated, e.g., by the short time from the beginning of the generation of the model and/or from the small variance in the readings that were used to generate the model), the processor may choose a specific method for transforming the location data. This may be, in some embodiments, a nearest neighbor approach. In some embodiments, it may a weighted average of the nearest neighbor approach and the approximation approach, but the weight of the approximation approach will be much lower than that of the nearest neighbor approach. In some embodiments, the location of the catheter may be determined based on a weighted average between two nearest neighbor estimates, each with a different nearest neighbor parameter. The two weights may be determined based on the low completeness of the model. For example, the weight of the location estimate based on the smaller nearest neighbor parameter may be much larger than the weight of the location estimate based on the larger nearest neighbor parameter.

After another 10 seconds, or any other period, e.g., a predetermined period, or a period based on how much new variance has been added to the readings, the model may be updated. In some embodiments, this is done by taking all the data collected so far (including the data used for generating the model and the data collected after the model was generated), and transforming it to generate a new model of the left atrium. The parameter indicative of the completeness of the model is adjusted (typically indicating a more complete model), and readings received from now on until the next model update, are shown on the present version of the model. Again, the received location data are transformed to locations in the updated model, based on the readings themselves, and a completeness indicator. Again, the completeness indicator may be used to determine weights for averaging various location estimates and/or for determining parameters in one or more transforms for generating a location estimate, and/or for determining approach for generating the location estimate. The locations determined (e.g., by averaging the different estimates based on the completeness indicator) are than shown on the updated model.

After another period, the model is again updated, and the locations are shown on the updated model. Once the model is updated, an indicator to its completeness is determined, and used for determining the location of the catheter based on location readings received after the model update. The determined location is shown on the updated model.

This way, the physician has a model that is updated as new substantial chunks of data are collected, while the estimated catheter location is updated on-line; e.g., at a frequency higher by at least a factor of 10 (in some embodiments by a factor of 100 or more) of the frequency at which the model is updated.

### General

It is expected that during the life of a patent maturing from this application many relevant intra-body probes will be developed; the scope of the term intra-body probe is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

1. A method of estimating a location of a catheter in a body part with respect to a model of the body part, the method comprising:
accessing a model generated based on location readings received from the catheter, the model representing the location readings as corresponding, by transformation, to model locations;
receiving current location readings from the catheter positioned in a location to be estimated from the current location readings;
determining a completeness indicator indicative of degree of change in model shape as location readings and corresponding model locations are added to the model; and
estimating the location to be estimated, wherein the estimating includes transforming the current location readings to a current location estimate using the completeness indicator to adjust transformation of the model location readings to model locations.

2. The method of claim 1, wherein the body part is a heart chamber.

3. The method of claim 1, wherein the completeness indicator is based on the time that elapsed from when the model generation started and the time the location readings were received from the location to be determined.

4. The method of claim 1, wherein the completeness indicator is based on variance in model location readings.

5. The method of claim 4, wherein the completeness indicator indicates greater completeness as overall variance in model location readings increases.

6. The method of claim 4, wherein the completeness indicator indicates greater completeness as a rate of change in overall variance in model location readings decreases.

7. The method of claim 6, wherein the indication of greater completeness as rate of change in overall variance in model location readings decreases is jointly dependent on a history of variance in recent location readings.

8. The method of claim 1, wherein the estimating the location to be estimated comprises calculating a similarity between current and model location readings depending on distance between the current and the location readings in a measurement space, and the calculation of similarity is adjusted based on the completeness indicator.

9. The method of claim 1, wherein the completeness indicator adjusts the transforming so that the more complete is the model, the larger is a distance between the current location readings and model location readings used in the estimating.

10. The method of claim 8, wherein the distance is Euclidean.

11. The method of claim 1, comprising:
providing a first estimate and a second estimate of the location to be estimated;
wherein the estimating uses the completeness indicator to adjust joint use of the first estimate and the second estimate to estimate the location to be estimated.

12. The method of claim 11, wherein the estimating comprises:
weighting the first estimate and the second estimate based on the completeness of the model indicated by the completeness indicator; and
averaging the first and second estimates according to the weights.

13. The method of claim 12, wherein at least one of the first estimate and the second estimate is based on an approximation approach, wherein
the approximation approach comprises transforming the current location readings to the location to be estimated using a transformation that approximates the transformation used to transform the model location readings to locations for generating the model.

14. The method of claim 12, wherein at least one of the first estimate and the second estimate is based on a nearest neighbor approach, wherein
the nearest neighbor approach comprises determining the location to be estimated based on locations assigned to model location readings similar to the current model readings, with similarity between given location readings being dependent on a distance between the given location readings in a measurement space and a nearest neighbor parameter.

15. The method of claim 14, wherein the first estimate is based on the nearest neighbor approach with a first nearest neighbor parameter, and the second estimate is based on the nearest neighbor approach with a second nearest neighbor parameter, different than the first nearest neighbor parameter.

16. The method of claim 11, wherein estimating the location comprises:
considering locations assigned to model location readings that are similar to the current location readings, wherein in generating the first estimate, similarity between location readings depends on Euclidean distance between the location readings in a measurement space; and in generating the second estimate, similarity between location readings depends on geodesic distance between the location readings in the same measurement space.

17. The method of claim 1, wherein estimating the location comprises selecting an estimation method based on the completeness indicator; and estimating the location using the selected estimation method.

18. The method of claim 1, wherein estimating the location comprises using an estimation method that depends upon a value of a parameter, and setting the value of the parameter based on the completeness indicator.

19. A method of guiding navigation of a catheter within a body part, the method comprising:
showing an estimated location of the catheter with respect to the body part, on a model of the body part, generated based on location readings received from the catheter, and representing the location readings as corresponding, by transformation, to model locations; and
updating the shown location as new location readings are received from the catheter;
wherein the location is estimated by:
receiving a completeness indicator indicative of degree of change in model shape as location readings and corresponding model locations are added to the model,
receiving current location readings from the catheter when the catheter is in the location to be estimated, and
estimating the location to be estimated, wherein the estimating includes transforming the current location readings to a current location estimate using the completeness indicator to adjust transformation of the model location readings to model locations.

20. The method of claim 19, wherein the completeness indicator is based on a time that lapsed from when the model generation has started and a time the current location readings were received.

21. The method of claim 19, wherein the completeness indicator is based on variance in model location readings.

22. The method of claim 19, wherein estimating the location comprises considering locations assigned to similar model location readings during the generation of the model, wherein similarity between location readings depends on distance between location readings in a measurement space.

23. The method of claim 19, wherein estimating the location comprises estimating based on model location readings and current location readings; and the more complete is the model, the larger is the distance between the current location readings and the model location readings used in the estimating.

24. The method of claim 22, wherein the distance is Euclidean.

25. The method of claim 19, comprising:
providing a first estimate and a second estimate of the location to be estimated; and
wherein the estimating uses the completeness indicator to adjust joint use of the first estimate and the second estimate to estimate the location to be estimated.

26. The method of claim 25, wherein the estimating comprises:
weighting the first estimate and the second estimate based on the completeness of the model indicated by the completeness indicator; and
averaging the first and second estimates according to the weights.

27. The method of claim 26, wherein the first estimate is based on an approximation approach and the second estimate is based on a nearest neighbor approach.

28. The method of claim 26, wherein the first estimate is based on a nearest neighbor approach with a first nearest neighbor parameter, and the second estimate is based on a nearest neighbor approach with a second nearest neighbor parameter, different than the first nearest neighbor parameter.

29. The method of claim 26, wherein estimating the location comprises:
considering locations assigned to model location readings during the generation of the model that are similar to the current location readings, wherein in generating the first estimate, similarity between location readings depends on Euclidean distance between the location readings in a measurement space; and in generating the second estimate, similarity between location readings depends on geodesic distance between the location readings in the same measurement space.

30. The method of claim 19, wherein the body part is a heart chamber.

31-55. (canceled)
